(19) United States
(12) Patent Application Publication
(10) Pub. No.: US 2025/0144058 A1
(43) Pub. Date: May 8, 2025

(54) PREVENTION OF VIRUS TRANSMISSION WITH APPLICATION OF A COMPOSITION

(71) Applicant: Solventum Intellectual Properties Company, Maplewood, MN (US)

(72) Inventors: Kevin D. Landgrebe, Woodbury, MN (US); Ranjani V. Parthasarathy, Woodbury, MN (US); Katie F. Wlaschin, St. Paul, MN (US)

(21) Appl. No.: 18/838,021

(22) PCT Filed: Feb. 13, 2023
(86) PCT No.: PCT/IB2023/051294

§ 371 Date: Aug. 13, 2024

Related U.S. Application Data
(60) Provisional application No. 63/268,493, filed on Feb. 25, 2022.

(51) Int. Cl. A61K 31/191; A61K 9/00; A61K 31/194; A61K 47/18; A61K 47/38; A61P 31/12

(52) U.S. Cl. CPC A61K 31/191; A61K 9/0043; A61K 31/194; A61K 47/18; A61K 47/38; A61P 31/12

(57) ABSTRACT

A method of preventing transmission of a virus from a mammalian subject. The method includes applying to a surface of the nasal cavity of the mammalian subject an aqueous composition. The aqueous composition includes a surfactant and a hydroxycarboxylic acid. The aqueous composition is at least substantially free of an antimicrobial agent. The aqueous composition has a viscosity of between 2,000 and 100,000 cps.

### BACKGROUND

Compositions and methods for preventing infection by or transmission of viruses have been described in, for example, U.S. Pat. App. Pub. 2017/0165296 (Goodall et al.), U.S. Pat. No. 5,466,680 (Rudy), and U.S. Pat. App. Pub. 2005/0232868 (Rennie et al.).

### BRIEF DESCRIPTION OF THE FIGURES

FIG. 1 is a schematic of animal pairs and cages for airborne and contact transmission experiments-water and food sources not shown.

### DETAILED DESCRIPTION

Viruses such as influenza, especially pandemic influenza, can cause significant morbidity and mortality and loss of productivity in the population. Influenza A has proven difficult to control using vaccination. On average, the annual vaccine is only 59% effective and is often significantly less so. Consequently, compositions and methods for limiting the transmission of viruses, including for Influenza A, are desirable.

Prior attempts of mitigating the impact of viruses include compositions that are intended to prevent infection (as opposed to preventing transmission) when sprayed into the nasal cavity by encapsulating, inactivating, or removing viruses from the nasal cavity to alleviate symptoms of the virus. Such compositions are directed for application shortly before or shortly after inoculation with a virus. It has been discovered, however, that certain, relatively viscous compositions (e.g., topical nasal sprays) having low pH can provide long-lasting reduction of virus transmission (and such reduction is observed even when applied long before or long after inoculation with the virus). Further, it has been discovered that such viscous, low-pH compositions can prevent virus transmission from a virus-infected animal to a virus-naïve animal even when the compositions do not substantially reduce the nasal viral load in the virus-infected animal from which transmission occurs.

Further, given the limited knowledge of the role of nasal infection in the transmission of influenza virus and that inoculation of mucosal tissue other than that in the nares (e.g., the mucosal surface of the eye) can cause respiratory symptoms of influenza, a number of viral transmission pathways can be envisioned. It has been surprisingly discovered, however, that treatment of the nasal mucosa, only, with the low-pH, relatively viscous compositions of the present disclosure can provide long-lasting protection against transmission of virus.

### Definitions

“Ambient temperature” refers to the temperature in the environment at which the method of the current disclosure is conducted. Typically, ambient temperature will be about 10° C. to about 30° C., and more particularly 15° C. to 25° C.

“Colonization” or “colonized” refers to having some presence of a virus whether asymptomatic, pre-symptomatic, or symptomatic.

“Effective amount” refers to the amount of the composition and/or component of a composition component that provides an activity that reduces or prevents transmission of a virus.

“Enveloped RNA virus” refers to an RNA virus that has a viral envelope. Examples of enveloped RNA viruses include flavivirus, alphavirus, togavirus, coronavirus, hepatitis D, orthomyxovirus (including influenza), paramyxovirus, rhabdovirus, bunyavirus, and filovirus.

“Film-forming” refers to a feature of a composition that when allowed to dry under ambient conditions (e.g., 23° C. and 50% relative humidity (RH)) on in-tact skin forms a continuous layer that does not flake off after simple flexing of the tissue.

“Substantive” refers to resistance of the continuous layer of the dried film-forming polymer to removal (e.g., rinsed off) of tissue (e.g., skin or a mucous membrane) due to the presence of water, blood, or other body fluids such as secreted mucous from a mucous membrane. “Infection” refers to the combination of the presence of the virus in a host and the host response to the virus. The infection can generally result in symptomatic responses from the host. “Influenza” refers to a disease caused by influenza viruses A, B, C, D. Influenza virus A includes at least H1N1, H2N2, H3N2, H5N1, H7N7, H1N2, H9N2, H7N2, H7N3, H10N7, H7N9, H6N1.

“Inoculation” refers to the act or process of introducing a pathogen such as a virus into a living organism. Inoculation can generally refer to exposing the mammalian subject to the virus either directly (via a solution applied using a pipette or syringe or via contact transmission from another animal) or indirectly or passively (including via airborne transmission from another animal). Inoculation occurs before the symptoms are observable in a mammalian subject.

“Interact” refers to contact transmission, or to airborne transmission, between at least two animal subjects, or to transmission of pathogen from a pathogen-laden-surface to an animal. “Mammalian subject” refers to humans, sheep, horses, cattle, pigs, dogs, cats, guinea pigs, ferrets, rats, mice, bats, or other mammal.

“Mucosal tissue” refers to the mucus-producing membranous surfaces of the nasal cavity (including anterior nares, nasopharynx, etc.), vagina, and other similar tissues. Examples include mucosal membranes such as nasal, rectal, urethral, ureteral, vaginal, cervical, and uterine mucosal membranes.

“Retain” refers to the action of continuing to hold a virus within or on mucosal tissue during normal physiological activity (e.g., breathing, sneezing, coughing, spitting). Retention can be by virtue of entrapment, agglomeration, intracellular presence of the virus, or combinations thereof. “Surfactant” refers to synthetic and naturally occurring amphiphilic molecules that have hydrophobic portion(s) and hydrophilic portion(s). Due to their amphiphilic (amphipathic) nature, surfactants typically can reduce the surface tension between two immiscible liquids, for example, the oil and water phases in an emulsion, stabilizing the emulsion. Surfactants can be characterized based on their relative hydrophobicity and/or hydrophilicity. For example, relatively lipophilic surfactants are more soluble in fats, oils and waxes, and typically have HLB (hydrophile-lipophile balance) values less than or about 10, while relatively hydrophilic surfactants are more soluble in aqueous compositions, for example, water, and typically have HLB values greater than or about 10.

“Symptom” refers to a physical or physiological feature indicating a condition or a disease. Symptoms of influenza can include runny nose, fever, aching muscles, headaches, chills, sweats, dry cough, fatigue, nasal congestion, sore throat, or combinations thereof. Viral loads of influenza at the onset of symptoms can be viral species-dependent and host species-dependent. For example, in a human, symptoms of influenza A can be present with a viral load of at least 100000 PFU/mL, at least 0.5 million PFU/mL, or at least 1 million PFU/mL. In a mouse, the viral load of influenza A can be present, at the onset of symptoms, of at least 100 PFU/mL.

“Thickener” refers to a substance that increases the viscosity of a liquid without substantially changing its other properties. Thickeners can be anionic, cationic, or neutral. The thickened compositions of the present disclosure do not include a cross-linked polymer (i.e., a gel).

“Treatment plan” refers to a sequence of treatments. “Treat” or “treatment” means to improve the condition of a subject relative to the affliction, typically in terms of clinical symptoms of the condition or viral- or bacterial load.

The terms “comprises” and variations thereof do not have a limiting meaning where these terms appear in the description and claims.

As used herein, “a,” “an,” “the,” “at least one,” and “one or more” are used interchangeably. The term “and/or” means one or all of the listed elements (e.g., preventing and/or treating an infection means preventing, treating, or both treating and preventing further infections).

Also herein, the recitations of numerical ranges by endpoints include all numbers subsumed within that range (e.g., 1 to 5 includes 1, 1.5, 2, 2.75, 3, 3.80, 4, 5, etc.).

In some embodiments, the present disclosure relates to compositions and methods of preventing transmission of a virus (e.g., an enveloped RNA virus). The method may include applying to a tissue of a first mammalian subject an aqueous composition that includes a surfactant and a hydroxycarboxylic acid.

In some embodiments, suitable surfactants for use in the compositions of the present disclosure may include nonionic surfactants, amphoteric surfactants, anionic surfactants, or a combination thereof.

In some embodiments, suitable surfactants for the compositions of the present disclosure include nonionic surfactants. It has been found that polyalkoxylated and, in particular, polyethoxylated nonionic surfactants can stabilize the film-forming polymers of the present disclosure in aqueous solutions particularly well. In general, useful polyalkoxylated nonionic surfactants may have a hydrophile/lipophile balance (HLB) of at least about 14, or at least about 16. Useful polyalkoxylated nonionic surfactants may have an HLB of no greater than about 19. When using combinations of nonionic surfactants, a weight average HLB is used to determine the HLB of the nonionic surfactant system. As used herein, the HLB is defined as one-fifth the weight percentage of ethylene oxide segments in the surfactant molecule.

Surfactants of the nonionic type that are useful in the compositions of the present disclosure include:
1. Polyethylene oxide extended sorbitan monoalkylates (i.e., Polysorbates), e.g., Polysorbate 20 commercially available as NIKKOL TL-10 (from Barret Products).
2. Polyalkoxylated alkanols. Surfactants such as those commercially available under the trade designation BRIJ from ICI Specialty Chemicals, Wilmington, Delaware having an HLB of at least about 14. In particular, BRIJ 78 and BRIJ 700, which are stearyl alcohol ethoxylates having 20 and 100 moles of polyethylene oxide, respectively, have proven useful in the compositions of the present disclosure as has ceteareth 55, which is commercially available under the trade designation PLURAFAC A-39 from BASF Corp., Performance Chemicals Div., Mt. Olive, N.J.3.
3. Polyalkoxylated alkylphenols. Useful surfactants of this type include polyethoxylated octyl or nonyl phenols having HLB values of at least about 14, which are commercially available under the trade designations ICONOL and TRITON, from BASF Corp., Performance Chemicals Div., Mt. Olive, N.J. and Union Carbide Corp., Danbury, Conn., respectively. Examples include TRITON X100 (an octyl phenol having 15 moles of ethylene oxide available from Union Carbide Corp., Danbury, Conn.) and ICONOL NP70 and NP40 (nonyl phenol having 40 and 70 moles of ethylene oxide units, respectively, available from BASF Corp., Performance Chemicals Div., Mt. Olive, N.J.). Sulfated and phosphated derivatives of these surfactants are also useful. Examples of such derivatives include ammonium nonoxynol-4-sulfate, which is commercially available under the trade designation RHODAPEX CO-436 from Rhodia, Dayton, N.J.
4. Polaxamers. Surfactants based on block copolymers of ethylene oxide (EO) and propylene oxide (PO) have been shown to be effective at stabilizing the film-forming polymers of the present disclosure and provide good wetting. EO-PO-EO blocks and PO-EO-PO blocks are useful surfactants for use in the compositions of the present disclosure provided the HLB is at least about 14 and preferably at least about 16. Such surfactants are commercially available under the trade designations PLURONIC and TETRONIC from BASF Corp., Performance Chemicals Div., Mt. Olive, N.J. It is noted that the PLURONIC surfactants from BASF have reported HLB values that are calculated differently from that as described above, and for these surfactants, the preferable HLB values refer to the HLB values reported by BASF. For example, preferred PLURONIC surfactants are L-64 and F-127, which have HLBs of 15 and 22, respectively.
5 Polyalkoxylated esters. Polyalkoxylated esters of glycols such as ethylene glycol, propylene glycol, glycerol, and the like may be partially or completely esterified, i.e., one or more alcohols may be esterified, for example, with a (C8-C22) alkyl carboxylic acid. Such polyethoxylated esters having an HLB of at least about 14, and preferably at least about 16, are suitable for use in compositions of the present disclosure.
6. Alkyl Polyglucosides. Alkyl polyglucosides, such as those described in U.S. Pat. No. 5,951,993 (Scholz et al.), starting at column 9, line 44, are compatible with the film-forming polymers of the present disclosure and may contribute to polymer stability. Examples include glucopon 425, which has a (C8-C16) alkyl chain length with an average chain length of 10.3 carbons and 1-4 glucose units.

In some embodiments, suitable surfactants for the compositions of the present disclosure include amphoteric surfactants. Suitable amphoteric surfactants may have tertiary amine groups which may be protonated as well as quaternary amine-containing zwitterionic surfactants. Amphoteric surfactants that are useful in the compositions of the present disclosure include:
1. Ammonium Carboxylate Amphoterics. This class of surfactants can be represented by the following formula:

R³—(C(O)—NH)ₐ—R⁵—N⁺(R⁴)₂—R⁶—COO
wherein: a=0 or 1; R³ is a (C7-C21) alkyl group (saturated straight, branched, or cyclic group), a (C6-C22) aryl group, or a (C6-C22) aralkyl or alkaryl group (saturated straight, branched, or cyclic alkyl group), wherein R³ may be optionally substituted with one or more N, O, or S atoms, or one or more hydroxyl, carboxyl, amide, or amine groups; R⁴ is H or a (C1-C8) alkyl group (saturated straight, branched, or cyclic group), wherein R⁴ may be optionally substituted with one or more N, O, or S atoms, or one or more hydroxyl, carboxyl, amine groups, a (C6-C9) aryl group, or a (C6-C9) aralkyl or alkaryl group; and R⁵ and R⁶ are each independently a (C1-C10) alkylene group that may be the same or different and may be optionally substituted with one or more N, O, or S atoms, or one or more hydroxyl or amine groups.
   In some embodiment, in the formula above, R³ is a (C1-C16) alkyl group, R⁴ is a (C1-C2) alkyl group preferably substituted with a methyl or benzyl group and most preferably with a methyl group. When R⁴ is H, it is understood that the surfactant at higher pH values could exist as a tertiary amine with a cationic counterion such as Na, K, Li, or a quaternary amine group.
   Examples of such amphoteric surfactants include certain betaines such as cocobetaine and cocamidopropyl betaine (commercially available under the trade designations MACKAM CB-35 and MACKAM L from Mcintyre Group Ltd., University Park, Ill.); monoacetates such as sodium lauroamphoacetate; diacetates such as disodium lauroamphoacetate; amino- and alkylamino-propionates such as lauraminopropionic acid (commercially available under the trade designations MACKAM IL, MACKAM 2L, and MACKAM 151L, respectively, from McIntyre Group Ltd.).
2. Ammonium Sulfonate Amphoterics. This class of amphoteric surfactants is often referred to as “sultaines” or “sulfobetaines” and can be represented by the following formula

R³—(C(O)—NH)ₐ—R⁵—N⁺(R⁴)₂—R⁶—SO₃⁻
wherein R³-R⁶ and “a” are define above. Examples include cocamidopropylhydroxysultaine (commercially available as MACKAM 50-SB from McIntyre Group Ltd.).

In some embodiments, suitable surfactants for the compositions of the present disclosure include anionic surfactants. Suitable anionic surfactants include:
1. Sulfonates and Sulfates. Suitable anionic surfactants include sulfonates and sulfates such as alkyl sulfates, alkylether sulfates, alkyl sulfonates, alkylether sulfonates, alkylbenzene sulfonates, alkylbenzene ether sulfates, alkylsulfoacetates, secondary alkane sulfonates, secondary alkylsulfates and the like. Many of these can be represented by the formulas:

R³—(OCH₂CH₂)ₙ(OCH(CH₃)CH₂)ₚ-(Ph)ₐ-(OCH₂CH₂)ₘ—(O)_{b}—SO₃⁻M⁺
and

R³—CH[SO₃-M⁺]-R⁷
wherein: a and b-0 or 1; n, p, m=0-100 (preferably 0-40, and more preferably 0-20); R³ is defined as above; R⁷ is a (C1-C12) alkyl group (saturated straight, branched, or cyclic group) that may be optionally substituted by N, O, or S atoms or hydroxyl, carboxyl, amide, or amine groups; Ph-phenyl; and M is a cationic counterion such as Na, K, Li, ammonium, a protonated tertiary amine such as triethanolamine or a quaternary ammonium group.
   In the formula above, the ethylene oxide groups (i.e., the “n” and “m” groups) and propylene oxide groups (i.e., the “p” groups) can occur in reverse order as well as in a random, sequential, or block arrangement. Preferably for this class, R³ comprises an alkylamide group such as R⁸—C(O)N(CH₃)CH₂CH₂— as well as ester groups such as —OC(O)—CH₂— wherein R⁸ is a (C8-C22) alkyl group (saturated branched, straight, or cyclic group).
   Examples include: alkyl ether sulfonates such as lauryl ether sulfates such as POLYSTEP B12 (n=3-4, M=sodium) and B22 (n=12, M=ammonium) available from Stepan Company, Northfield, Ill. and sodium methyl taurate (available under the trade designation NIKKOL CMT30 from Nikko Chemicals Co., Tokyo, Japan); secondary alkane sulfonates such as Hostapur SAS which are Sodium (C14-C17) secondary alkane sulfonates (alpha-olefin sulfonates) available from Clariant Corp., Charlotte, N.C.; methyl-2-sulfoalkyl esters such as sodium methyl-2-sulfo (C12-16) ester and disodium 2-sulfo (C12-C16) fatty acid available from Stepan Company under the trade designation ALPHASTE PC-48; alkylsulfoacetates and alkylsulfosuccinates available as sodium laurylsulfoacetate (under the trade designation LANTHANOL LAL) and disodiumlaurethsulfosuccinate (STEPANMILD SL3), both from Stepan Company; alkylsulfates such as ammoniumlauryl sulfate commercially available under the trade designation STEPANOL AM from Stepan Company.
2. Phosphates and Phosphonates. Suitable anionic surfactants also include phosphates such as alkyl phosphates, alkylether phosphates, aralkylphosphates, and aralkylether phosphates. Many may be represented by the formula:

[R³-(Ph)ₐ-O(CH₂CH₂O)ₙ(CH₂CH(CH₃)O)ₚ]_{q}—P(O)[O⁻M⁺]ᵣ
where: Ph, R³, a, n, p, and M are defined above; r is 0-2 and q=1-3, with the proviso that when q=1, r=2, and when q=2, r=1, and when q=3, r=0. As above, the ethylene oxide groups (i.e., the “n” groups) and propylene oxide groups (i.e., the “p” groups) can occur in reverse order as well as in a random, sequential, or block arrangement.
   Examples include a mixture of mono-, di- and tri-(alkyltetraglycolether)-o-phosphoric acid esters generally referred to as trilaureth-4-phosphate commercially available under the trade designation HOSTAPHAT 340KL from Clariant Corp., as well as PPG-5 ceteth 10 phosphate available under the trade designation CRODAPHOS SG from Croda Inc., Parsipanny, N.J.
3. Amine Oxides. Suitable anionic surfactants also include amine oxides including alkyl and alkylamidoalkyldialkylamine oxides of the following formula:

(R³)₃—N→O
wherein R³ is defined above, and each R³ may be the same or different. Optionally, the R³ groups can be joined to form a heterocyclic ring with the nitrogen to form surfactants such as amine oxides of alkyl morpholine, alkyl piperazine, and the like. Preferably two R³ groups are methyl and one R³ group is a (C12-C16) alkyl or alkylamidopropyl group.
   Examples of amine oxide surfactants include those commercially available under the trade designations AMMONYX LO, LMDO, and CO, which are lauryldimethylamine oxide, laurylamidopropyldimethylamine oxide, and cetyl amine oxide, all from Stepan Company.

In some embodiments, combinations of various surfactants can be used. For example, in some embodiments, a combination of nonionic surfactants and anionic surfactants can be employed.

In some embodiments, it may be desirable to select one or more surfactants that associate or potentially associate with other components in the composition after dry-down to facilitate improved toleration. In some embodiments, certain anionic surfactants such as methyl-2-sulfoalkyl esters (e.g., sodium methyl-2-sulfo (C12-16) ester and disodium 2-sulfo (C12-C16) fatty acid available from Stepan Company under the trade designation ALPHASTEP PC-48) in combination with polyamine oxide film-forming polymers may increase the substantivity of a dried film of the composition.

In some embodiments, the surfactants employed may have low inorganic salt impurities such as sodium chloride, sodium sulfate, and the like. In this regard, such salt content may be sufficiently low such that a 20% solution of the surfactant in water has a conductivity of less than 100 micromhos/cm, less than 85 micromhos/cm, or less than 75 micromhos/cm.

In some embodiments, surfactants may be present in the composition in an amount of between 0.1 wt % and 10 wt %, between 0.1 wt % and 7 wt %, between 0.1 wt % and 5 wt %, between 0.5 wt % and 4 wt %, or between 0.1 wt % and 3 wt %, based on the total weight of the composition. Generally, too little surfactant may result in an unstable composition, and too much surfactant may undermine the substantivity of the dried composition on skin.

In some embodiments, the compositions of the present disclosure may be buffered to prevent pH drift during storage. Generally, for use in the nasal cavity, the composition may become irritating if its pH is lower than about 2. In this regard, in some embodiment, the compositions of the present disclosure may have a pH that is 2.5 to 4.5, 3.0 to 4.5, 3.0 to 4.0, or 3.0 to 3.5.

In some embodiments, suitable hydroxycarboxylic acid buffers may include one or more compounds represented by the formula:

R¹(CR²OH)ₙ(CH₂)ₘCOOH

wherein: R¹ and R² are each independently H or a (C1-C8) alkyl group (saturated straight, branched, or cyclic group), a (C6-C12) aryl, or a (C6-C12) aralkyl or alkaryl group (saturated straight, branched, or cyclic alkyl group), wherein R¹ and R² may be optionally substituted with one or more carboxylic acid groups; m=0 or 1-5; and n=1-5 or 1-2.

In some embodiments, the hydroxycarboxylic acid buffers of the present disclosure may include beta- and alpha-hydroxy acids (BHAs, AHAs, respectively, collectively referred to as hydroxy acids (HAs)), salts thereof, lactones thereof, and/or derivatives thereof. These may include mono-, di-, and tri-functional carboxylic acids. In some embodiments, HAs having 1 or 2 hydroxyl groups and 1 or 2 carboxylic acid groups may be employed. Suitable HAs may include lactic acid, malic acid, citric acid, 2-hydroxybutanoic acid, 3-hydroxybutanoic acid, mandelic acid, gluconic acid, tartaric acid, salicylic acid, as well as derivatives thereof (e.g., compounds substituted with hydroxyls, phenyl groups, hydroxyphenyl groups, alkyl groups, halogens, as well as combinations thereof)). In some embodiments, HAs may include lactic acid, malic acid, or citric acid. These acids may be in D, L, or DL form and may be present as free acid, lactone, or salts thereof. Other suitable HAs are described in U.S. Pat. No. 5,665,776 (Yu et al.). Various combinations of hydroxycarboxylic acids can be used.

In some embodiments, suitable hydroxycarboxylic acids may include organic acids that feature at least one alcoholic hydroxyl and one carboxyl group in the molecule, such as: glycolic acid, gluconic acid, malic acid, lactic acid, tartaric acid, citric acid, hydroxybutyric acid, glyceric acid, tartronic acid, salicylic acid, m-hydroxybenzoic acid, p-hydroxybenzoic acid, gallic acid, mandelic acid, tropic acid, and like aromatic hydroxycarboxylic acids or mixtures thereof.

In some embodiments, the hydroxycarboxylic acid may also include substituted carboxylic acids. The substituted carboxylic acids may include diacids and triacids as well. The substituted carboxylic acid may be substituted with amino groups, keto groups, aldehyde groups, and mixtures thereof. In some embodiments, the hydroxycarboxylic acid has 8 carbon atoms or fewer, or 6 carbon atoms or fewer.

While typical buffered compositions have a buffer concentration of about 0.1 wt-% to about 2 wt-%, in some embodiments, the compositions of the present disclosure may include certain hydroxycarboxylic acids as buffers that can be used in much higher buffer concentrations. In this regard, in some embodiments, the hydroxycarboxylic acid buffer may be present in an amount of at least 2.5 wt-%, at least 3.5 wt-%, at least 5 wt-%, at least 7 wt-%, or at least 10 wt-%; and in an amount of no more than 15 wt-% or no more 10 wt-%, based on the total weight of the composition. In some embodiments, the hydroxycarboxylic acid buffer may be present in the composition in an amount of between 0.1 wt % and 10 wt %, between 0.1 wt % and 7.5 wt %, or between 1 wt % and 5 wt %, based on the total weight of the composition.

It may also be convenient in some applications to supply concentrates that have much higher concentration of hydroxycarboxylic acid buffer but when diluted to the use concentration the concentration of the hydroxycarboxylic acid buffer falls to within the specified ranges.

In some embodiments, water may be present in the composition in an amount of between 65 wt % and 95 wt %, between 75 wt % and 95 wt %, or between 80 wt % and 90 wt %, based on the total weight of the composition.

In some embodiments, the compositions of the present disclosure may be at least substantially free of antimicrobial agents. For purposes of the present disclosure, compositions are at least substantially free of antimicrobial agents if (i) they do not include an antimicrobial agent or (ii) they include antimicrobial agent(s) in an amount such that the difference in antimicrobial activity of the composition with the antimicrobial agent(s) relative to the composition without the antimicrobial agent(s) is less than 3 logs kill, 2 logs kill, or 1 log kill. For purposes of the present application, antimicrobial agents are cationic antiseptics such as chlorhexidine salts, cetylpyridinium chloride, octenidine salts, benzalkonium chloride, and polyhexamethylene biguanide; parachlorometaxylenol (PCMX); triclosan; hexachlorophene; phenols; hydrogen peroxide; silver and silver salts such as silver chloride; silver oxide and silver sulfadiazine; and iodine and its complexed forms, which are commonly referred to as iodophors. For purposes of the present disclosure, antimicrobial activity is as determined in accordance with the Time-Kill test of ASTM E2315 using an exposure time of 5 minutes of the microbe Staphylococcus aureus (ATCC 6538) to the composition.

In some embodiments, the compositions of the present disclosure can allow for microbial (e.g., bacteria, fungal) growth. In this regard, in some embodiments, when subjected to the Time-Kill test, the compositions may demonstrate a log reduction of microbes no greater than 3 logs, no greater than 2 logs, or no greater than 1 log.

In some embodiments, the compositions of the present disclosure may include a film-forming polymer, which is intended to improve substantivity of the composition (i.e., resistance of the continuous layer of the dried film-forming polymer to wash off of skin or be removed from a mucous membrane due to the presence of water, blood, or other body fluids such as secreted mucous from a mucous membrane.

In some embodiments, suitable film-forming polymers are substantive and resist removal by prolonged exposure to fluids such as water, saline, and body fluids such as mucous. The film-forming polymers may be nonionic, anionic, or cationic. In some embodiments, they may exhibit pressure sensitive adhesive properties. These include both synthetic and natural polymers as well as derivatives of natural polymers.

In some embodiments, the film-forming polymers are cationic. Surprisingly, the solubility and stability of cationic film-forming polymers are not negatively impacted by the presence of multifunctional carboxylic acid-containing hydroxyacids such as citric acid, malic acid, tartaric acid, and the like. This is particularly surprising since it would be expected that adding these acids to compositions containing cationic polymers at very high concentrations would result in precipitation of the polymer due to, for example, ionic crosslinking. Examples of suitable film-forming cationic polymers are those that include side-chain functional amine groups such as protonated tertiary amines, quaternary amines, amine oxides, and combinations thereof. In some embodiments, the film-forming cationic polymers may be as described U.S. Pat. No. 6,838,078, which is herein incorporated by reference in its entirety.

For certain preferred film-forming polymers, the amine group-containing monomers can be used to prepare the film-forming polymers in an amount of at least about 15 wt-%, more preferably at least about 20 wt-%, even more preferably at least about 25 wt-%, and most preferably at least about 30 wt-%, based on the total weight of the polymerizable composition (and preferably, based on the total weight of the polymer). The amine group-containing monomers used to prepare the film-forming polymers are typically used in an amount of no greater than about 70 wt-%, preferably no greater than about 65 wt-%, more preferably no greater than about 60 wt-%, and most preferably no greater than about 55 wt-%, based on the total weight of the polymerizable composition (and preferably, based on the total weight of the polymer).

The equivalent weight of the amine group contained in the polymer is preferably at least about 300, more preferably at least about 350, even more preferably at least about 400, and most preferably at least about 500, grams polymer per equivalent of amine group. The equivalent weight of the amine group contained in the polymer is preferably no greater than about 3000, more preferably no greater than about 1500, even more preferably no greater than about 1200, and most preferably no greater than about 950, grams polymer per equivalent of amine group.

In some embodiments, film-forming polymers may be present in the composition of the present disclosure in a total amount of at least 0.5 wt-%, at least 0.75 wt-%, or at least 1 wt-%; and no greater than 10 wt-% or no greater than 8 wt-%, based on the total weight of the composition. In some embodiments, film-forming polymers may be present in an amount sufficient to provide a substantive composition.

In some embodiments, in order to ensure adequate substantivity, the weight ratio of film-forming polymer to hydroxycarboxylic acid is at least about 0.5:1, at least 0.33:1, at least about 0.25:1, or at least about 0.20:1.

In some embodiments, the compositions can also include components such as, for example, organic solvents, hydrophobic components (e.g., petrolatum and oils), hydrophilic components (glycerin and various ether and/or polyether compounds), silicones, carbohydrates (polysaccharides such as hydroxypropyl methyl cellulose), thickeners such as carbopol, film-formers, emulsifiers, water, organic solvents (e.g., alcohols and polyols), stabilizers (e.g., polymers), fillers (e.g., organic materials such as polymeric particles and inorganic materials including ceramic particles, silica particles, clay particles, and glass particles), emollients/moisturizers, tonicity adjusting agents, chelating agents, anti-inflammatory agents, gelling agents, preservatives, pH adjusting agents, viscosity builders, time-release agents, dyes, fragrances or oils, and the like.

In some embodiments, thickeners, such as hydroxypropyl methylcellulose, can be employed to increase the viscosity of the compositions. In general, the polymers useful as thickeners have sufficient molecular weight to achieve thickening at generally less than 5 wt-% polymer, but not too high that the composition feels slimy and stringy. While the composition of the polymer will dramatically affect the molecular weight at which sufficient thickening will occur, the polymers may have a molecular weight of at least 250,000 daltons, or at least 500,000 daltons. The polymers may have a molecular weight of no greater than 3,000,000 daltons or no greater than 1,000,000 daltons.

Polymers used to thicken solutions can be classified as soluble, swellable, or associative in the aqueous compositions. Some polymers may fall into one or more of these classes. For example, certain associative polymers can be soluble in the aqueous system. Whether they are considered soluble, swellable, or associative in the aqueous system, suitable polymers may be film forming or not. Film forming polymers may retain the active virulence suppression component at the afflicted site for longer periods of time. This may be desirable for certain applications. For example, some film forming polymers may produce compositions that could not be easily washed off with water after being applied and dried.

As used herein, a soluble polymer is one that in dilute solution (i.e., 0.01-0.1 wt-% in the desired aqueous solvent system-defined as containing water and any other hydrophilic compounds), after heating for a sufficient time to ensure solubilization of any potentially soluble components, has no significant observable particles of greater than 1 micron in particle size, as determined by light scattering measurements using, for example, Malvern Masterisizer E Laser Particle Size Analyzer available from Malvern Co., Boston, Mass.

As used herein, a swellable polymer is one that in dilute solution (i.e., 0.01-0.1 wt-% in the desired aqueous solvent system), after heating for a sufficient time to ensure solubilization of any potentially soluble components, has a significant (i.e., detectable) number of observable particles of greater than 1 micron in particle size, as determined by light scattering measurements using, for example, Malvern Masterisizer E Laser Particle Size Analyzer.

As previously discussed, it has been discovered that, at least in part, the (relatively high) viscosity of the compositions of the present disclosure contribute to the desirable virus transmission mitigation properties of the composition. In this regard, the composition may have a viscosity of between 2,000 and 100,000 cps, between 3,000 and 50,000, or between 4,000 and 20,000 cps.

In some embodiments, the compositions of the present disclosure can be administered and/or applied in various formulations such as a spray, gel (e.g., cellulosic gel), lotion, ointment, solution, emulsion, dispersion, foam, coating, paste, powder, tablet, capsule, film, or the like.

For some applications, it is desirable that the compositions remain in a location where they are administered and/or applied. Such compositions are usually formulated to have a suitably high viscosity and/or to include a hydrophobic component that will enhance retention of the composition at the application location. These formulations can be, for example, an emulsion, ointment, gel, or lotion. Emulsions can be oil-in-water or water-in-oil.

In some embodiments, the compositions can be administered and/or applied locally. For example, the compositions can be applied to the nares of a mammal using a spray, or first applied to a swab, cloth, sponge, nonwoven wipe, paper product such as a tissue or paper towel, and then applied to the nares. When applied locally, the composition may remain where it was applied. That is, the composition may persist at the location for a desired period.

In some embodiments, the methods of the present disclosure may include applying an effective amount of the above-described composition to the mucosal tissue of either or both of a first mammalian subject not substantially colonized with a virus (e.g., an enveloped RNA virus) and a second mammalian subject that is substantially colonized with the virus, thereby preventing transmission of the virus from the second mammalian subject to the first mammalian subject upon interaction of the first and second mammalian subjects. In some embodiments, the mucosal tissue can be in the nasopharynx, oropharynx, nasal cavity, sinuses, or anterior nares of the mammalian subject. In some embodiments, the mucosal tissue can be in the nasal cavity.

In another aspect, a treatment plan can include applying the above-described composition to the (first and/or second) mammalian subject multiple times (i.e., multiple applications).

In another aspect, the application of the composition to the first or second mammalian subject can include applying the composition to the first and/or second mammalian subject at any time period (e.g., at least 1 hour, at least 4 hours, at least 8 hours, at least 24 hours, or greater than 24 hours) before the interaction with the other.

In some embodiments, the composition of the present disclosure can be applied according to a treatment plan. The treatment plan can include options for prophylactic treatment after and/or before inoculation with the virus. For example, in one treatment plan, the composition can be applied to the mammalian subject before inoculation (e.g., via the nares of the mammalian subject). In another treatment plan, the composition can be applied after inoculation with the virus (e.g., at the onset of any symptoms). In another treatment plan, the composition can be applied in response to another mammalian subject exhibiting symptoms (i.e., prophylaxis) to prevent transmission of the virus to the mammalian subject from the mammalian subject exhibiting symptoms. In another treatment plan, the composition can be applied to one or more mammalian subject(s) exhibiting symptoms and to one or more mammalian subjects that are in direct contact and/or indirect (via air flow) contact with the mammalian subject(s) exhibiting symptom(s).

In some embodiments, the treatment plan can occur over a duration. The duration can be at least 6 hours, at least 1 day, at least 3 days, at least 5 days, or at least 7 days, at least 14 days, at least 21 days, or at least 28 days. The treatment plan can include multiple applications of the compositions during the duration. The frequency of treatment can range between once every 72 hours to between 1 and 12 times per day. For example, the frequency of treatment can be once every 72 hours, once every 60 hours, once every 48 hours, once every 36 hours, once every 25 hours, once every 24.5 hours (i.e., more than 24 hours between applications), once every 24 hours, once every 12 hours, once every 8 hours, once every 6 hours, or 5 times-, 6 times-, 7 times-, or 8 times per day. The frequency of treatment can occur roughly evenly throughout the day.

In some embodiments, the treatment plan can include applying the composition to mucosal tissue of a mammalian subject. The application of the pharmaceutical composition can include inserting a swab with the composition impregnated therein into the nasal cavity and spreading the composition along the perimeter of the nasal cavity. In some embodiments, the composition can be applied to the nares at any depth. For example, the composition can be applied to the posterior nares at a depth of at least 1 cm from the tip of the nose.

### EXAMPLES

These examples are for illustrative purposes only and are not meant to limit the scope of the appended claims. All parts, percentages, ratios, etc. in the examples and the rest of the specification are by weight, unless noted otherwise. Materials used in the Examples and their sources are provided in Table 1. Solvents and other reagents used were obtained from MilliporeSigma, St. Louis, MO, unless otherwise noted.

TABLE 1
Materials

| | Designation or | |
|---|---|---|
| Material | Alternative Name | Source (Location) |
| Lactic Acid | | Millipore Sigma |
| Malic Acid | | Millipore Sigma |
| Hetoxyl Sta-100 | Polyoxyethylene(100) stearyl | Globe Seven (Columbus, |
| | ether | OH) |
| Crodafos SG | PPG-5-Ceteth-10-Phosphate | Croda, Inc. (Edison, NJ) |
| Ammonyx LMDO | Lauramidopropylamine/ | Stepan Company |
| | myristamidopropylamine oxide | (Northfield, IL) |
| | (30%-33$) | |
| Celquat SC-230M | Polyquaternium-10 | National Starch and |
| | | Chemical Co., |
| | | Bridgewater, NJ |
| Xylitol | | Millipore Sigma |
| Phosphate-buffered saline, | PBS | Thermo Fisher Scientific |
| pH = 7.4 | | (Waltham, MA) |
| A/Panama/2007/1999 | Pan99 | bei Resources |
| (H3N2) Influenza A Virus | | (Manassas, VA) |
| Ketamine | | Covetrus |
| | | (Dublin, OH) |
| Xylazine | | Covetrus |
| | | (Dublin, OH) |
| Madin-Darby Canine Kidney | MDCK-SIAT1 | Millipore Sigma/European |
| cells | | Collection of Authenticated |
| | | Cell Cultures |

### Test Methods

Viscosity was measured at ambient temperature (approximately 22° C.) and ambient pressure using a Brookfield DV2T viscometer. All samples were allowed to equilibrate at approximately 22° C. for 24 hours prior to measurement. The LV-04 (64) spindle and speed of 30 rpm for 1 minute were used for testing. In all cases, the sample size and container geometry were chosen to ensure that there were no “wall effects,” i.e., that the viscosity value was not affected by the container (a measurement not affected by wall effects is essentially equivalent to the viscosity measurement hypothetically taken in an infinitely large container).

Treatment and control compositions: In the following examples, the “treatment,” a viscous low-pH composition, comprised lactic acid (5% w/w), malic acid (2% w/w), surfactants (Hetoxyl Sta-100 (1.4% w/w), Crodafos SG (1% w/w), Ammonyx LMDO (0.75% w/w)), xylitol (10.0% w/w), and thickener (Celquat SC-230M (1% w/w), with the balance being water. The viscosity of the treatment composition was measured to be 5,180 cps and the pH was measured to be 3.2. The “control” used in the examples was phosphate-buffered saline (PBS), pH=7.4, commercially available from Thermo Fisher Scientific (catalog number 10010023).

Animals: Five-to six-week-old female Hartley strain guinea pigs were obtained disease-free from Charles River Laboratories (Wilmington, MA). Animals weighed between 350 and 400 grams (g). Animals were allowed access to food and water ad libitum and were kept on a 12-hour light-dark cycle.

Infection and monitoring of guinea pigs: Guinea pigs were anesthetized with a 175-microliters (μl) mixture of ketamine (30 mg/kg of body weight) and xylazine (5 mg/kg) administered intramuscularly. An inoculum of “Pan99” influenza A virus was instilled intranasally by applying a volume of a stock solution of influenza virus (3×104 plaque forming units per milliliter (PFU/mL) A/Panama/2007/1999 (H3N2)) to each naris.

Collection of guinea pig nasal wash samples: Prior to nasal wash sample collection, guinea pigs were anesthetized as described above. Nasal washing was performed by instilling a total of 1 mL of phosphate-buffered saline (PBS) into the nares of a guinea pig and allowing it to drain into a sterile petri dish. Samples were stored at −80° C. before analysis by plaque assay.

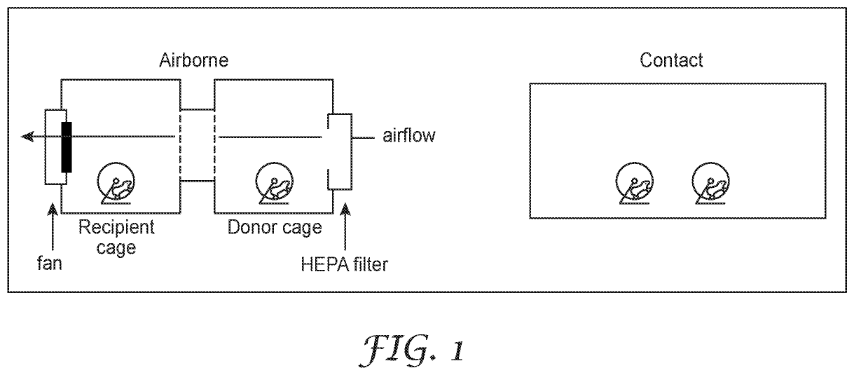

Experiments: Animals were grouped in pairs in the experiments. Each animal was identified as one animal of a pair that consisted of an influenza virus-naïve “recipient” animal and an influenza virus-inoculated “donor” animal. The experimental arrangements for the airborne transmission and contact transmission experiments are shown in FIG. 1.

### Example 1: Prevention of Airborne Transmission of Influenza Virus

In Example 1:
12 animals were designated “untreated donor,” indicating that those animals were not to be treated with anything during the experiment,
4 animals were designated “PBS control donor,” indicating that those 4 donor animals were to receive treatment using the PBS control, and
8 animals were designated “viscous, low-pH recipient,” indicating that those animals were to be treated with the viscous, low-pH composition,
4 animals were designated “untreated recipient,” indicating that those animals were not to be treated with anything during the experiment, and
4 animals were designated “PBS control recipient,” indicating that those animals were to be treated with PBS control during the experiment.

Groups of animal pairs for Example 1 are shown in Table 2.

TABLE 2
Pairings Of Animals For Example 1 (Airborne Transmission)

| Pair # | Donor treatment | Recipient treatment |
|---|---|---|
| 1-8 | No treatment | Viscous, low-pH composition |
| 9-12 | No treatment | No treatment |
| 13-16 | PBS control | PBS control |

On Day “−1,” the donor animals in pairs 13-16 were treated with PBS control by instilling 50 microliters PBS into each naris in an awake, upright animal.

On Day “0,” all donor animals (donor animals of pairs 1-16) animals were inoculated under ketamine anesthesia (as described above) intranasally with 75 microliters of “Pan 99” influenza virus per naris (as described above). Three hours later, awake recipients in pairs 1-8 were treated with the viscous, low-pH composition, and the awake animals in pairs 13-16 (the donors and the recipients in pairs 13-16) were treated using the PBS control.

On days +1, +3, +5 and +7, all animals were anesthetized, underwent a nasal wash (by instilling 1 mL PBS sampling solution into the nares (while prone), were allowed to awaken and then the recipient animals in pairs 1-8 were treated with the viscous low-pH composition, and the donor animals and recipient animals were treated with PBS control.

On days +2, +4, and +6, the recipient animals in pairs 1-8 were treated with the viscous, low-pH composition, and the donor animals and recipient animals in pairs 13-16 were treated with the PBS control.

Nasal washes collected on days +1, +3, +5, and +7 were diluted 1-to-10 with PBS immediately after collection. (Earlier in vitro experiments suggested that a 1-to-10 dilution was adequate to prevent the viscous low-pH composition from inactivating virus in the nasal wash samples prior to titration by plaque assay.)

For quantitation of viable virus, nasal wash samples were serially diluted and plated onto Madin-Darby Canine Kidney (MDCK) cells to quantify virus. Virus quantities were reported as PFU/mL. Virus quantities above about 30 PFU/mL are reported as a transmission event. “#transmission events” indicates number of recipient animals with at least 1 positive viral culture (>30 PFU/mL) between days 1 and 7.

The results of the transmission experiment are shown in Table 3.

TABLE 3
Results Of Prevention Of Airborne Transmission Of Influenza
Virus Expressed As Number Of Transmission Events

| | Donor | | Result (# of |
|---|---|---|---|
| Pair # | treatment | Recipient treatment | transmission events) |
| 1-8 | No treatment | Viscous, low-pH composition | 1 |
| 9-12 | No treatment | No treatment | 4 |
| 13-16 | PBS control | PBS control | 2 |

The results of Example 1 show that ⅛ (12.5%) of the transmission pairs in which only the recipients were treated with the viscous, low-pH composition acquired the influenza virus by airborne transmission. In contrast, all four (100%) of the untreated recipients acquired the virus by transmission, and in the pairs 13-16 in which donors and recipients were treated with the control, 50% of the recipients acquired the virus by transmission from an inoculated donor.

### Example 2: Prevention Of Contact Transmission Of Influenza Virus

In Example 2:
4 animals were designated “untreated donor,” indicating that those animals were not to be treated with anything during the experiment,
4 animals were designated “viscous, low-pH recipient,” indicating that those animals were to be treated with the viscous, low-pH composition,
Groups of animal pairs for Example 2 are shown in Table 4.

TABLE 4
Pairings Of Animals For Example 2 (Contact Transmission)

| Pair # | Donor treatment | Recipient treatment |
|---|---|---|
| 1-4 | No treatment | Viscous, low-pH composition |

On Day “0,” all donor animals were inoculated under ketamine anesthesia (as described above) intranasally with 75 microliters of “Pan 99” influenza virus per naris (as described above). Three hours later, awake recipients in pairs 1-4 were treated with the viscous, low-pH composition.

On days +1, +3, +5 and +7, all animals were anesthetized, underwent a nasal wash (by instilling 1 mL PBS sampling solution into the nares (while prone), were allowed to awaken and then the recipient animals in pairs 1-4 were treated with the viscous low-pH composition.

On days +2, +4, and +6, the recipient animals in pairs 1-4 were treated with the viscous, low-pH composition.

Nasal washes collected on days +1, +3, +5, and +7 were diluted 1-to-10 with PBS immediately after collection.

For quantitation of viable virus, nasal wash samples were serially diluted and plated onto MDCK cells to quantify virus. Virus quantities were reported as plaque-forming units (pfu). Virus quantities above about 30 PFU/mL are reported as a transmission event. “#transmission events” indicates number of recipient animals with at least 1 positive viral culture (>30 PFU/mL) between days 1 and 7.

The results of the transmission experiment are shown in Table 5.

TABLE 5
Results Of Prevention Of Contact Transmission Of Influenza
Virus Expressed As Number Of Transmission Events

| | Donor | | Result (# of |
|---|---|---|---|
| Pair number | treatment | Recipient treatment | transmission events) |
| 1-4 | No treatment | Viscous, low-pH | 0 |
| | | composition | |

The results of Example 2 show that none (0%) of the influenza virus-naïve recipients acquired the influenza virus from an inoculated donor housed in the same cage as each recipient.

### Example 3: Post-Exposure Treatment to Prevent Contact Transmission of Influenza Virus

In Example 3:
12 animals were designated “untreated donor,” indicating that those animals were not to be treated with anything during the experiment,
8 animals were designated “viscous, low-pH recipient,” indicating that those animals were to be treated with the viscous, low-pH composition,
4 animals were designated “PBS control recipient,” indicating that those animals were to be treated with PBS control during the experiment.

Groups of animal pairs for Example 3 are shown in Table 6.

TABLE 6
Pairings Of Animals For Example 3 (Contact Transmission)

| | Donor | |
|---|---|---|
| Pair number | treatment | Recipient treatment |
| 1-8 | No treatment | Viscous, low-pH composition |
| 9-12 | No treatment | PBS control |

On Day “0,” all donor animals were inoculated under ketamine anesthesia (as described above) intranasally with 75 microliters of “Pan 99” influenza virus per naris (as described above). Three hours later, each recipient in pairs 1-8 and in pairs 9-12 was placed in the same cage with the awake donor animal of its pairing and left undisturbed for 24 hours.

On days +1, +3, +5 and +7, all animals were anesthetized, underwent a nasal wash (by instilling 1 mL PBS sampling solution into the nares (while prone), were allowed to awaken and then the recipient animals in pairs 1-8 were treated with the viscous low-pH composition, and the recipient animals in pairs 9-12 were treated with PBS control.

On days +2, +4, and +6, the recipient animals in pairs 1-8 were treated with the viscous, low-pH composition, and the recipient animals in pairs 9-12 were treated with PBS control.

Nasal washes collected on days +1, +3, +5, and +7 were diluted 1-to-10 with PBS immediately after collection.

For quantitation of viable virus, nasal wash samples were serially diluted and plated onto MDCK cells to quantify virus. Virus quantities were reported as plaque-forming units (pfu). Virus quantities above about 30 PFU/mL are reported as a transmission event. “#transmission events” indicates number of recipient animals with at least 1 positive viral culture (>30 PFU/mL) between days 1 and 7.

The results of the transmission experiment are shown in Table 7.

TABLE 7
Results Of Post-Exposure Treatment To Prevent
Contact Transmission Of Influenza Virus Expressed
As Number Of Transmission Events

| | | | Results (# of |
|---|---|---|---|
| Pair # | Donor treatment | Recipient treatment | transmission events) |
| 1-8 | No treatment | Viscous, low-pH | 1 |
| | | composition | |
| 9-12 | No treatment | PBS control | 4 |

The results of Example 3 show that ⅛ (12.5%) of the transmission pairs in which only the recipients were treated with the viscous, low-pH composition acquired the influenza virus by contact transmission. In contrast, all four (100%) of the recipients treated with the PBS control acquired the virus by transmission.

The results from the examples show that the viscous, low-pH composition is effective at preventing transmission of influenza virus even when virus-naïve recipients are exposed to inoculated donors for 24 hours prior to treatment using the viscous, low-pH composition of the present disclosure.

### Example 4: Assessment of Nasal Viral Load in Treated Donors

In Example 4, donor animals were treated with the same viscous, low-pH composition of the disclosure that was used to treat animals in examples 1-3.

On day “−1,” four guinea pigs were treated with viscous, low-pH composition by instilling 50 mL of the composition into each naris (100 IL per guinea pig) in awake, upright animals.

On Day “0,” all four animals were inoculated under ketamine anesthesia (as described above) intranasally with 75 microliters of “Pan 99” influenza virus per naris (as described above).

Six hours later, all animals had awoken and were treated with the viscous, low-pH composition as described above for day “−1.”

On Days +1, +3, +5 and +7: All guinea pigs were anesthetized, underwent a nasal wash (1 mL PBS sampling solution), and then were allowed to awaken. On days 1, 3, and 5, after awakening, the guinea pigs were treated with the low-pH viscous composition as described above (day −1).

On Days 2, 4, 6: All guinea pigs were treated with the low-pH viscous composition, as described above (day −1).

Nasal washes were plated onto MDCK cells for quantitation of viable virus as discussed above for Example 1.

The results of Example 4 are shown in Table 8.

TABLE 8
Results Of Assessment Of Nasal Viral Load
In Treated Donors (Expressed In PFU/Ml)

| | Day 1 | Day 3 | Day 5 | Day 7 |
|---|---|---|---|---|
| Average viral load | 63,100 | 63,100 | 7,940 | 100 |
| (PFU/mL) | | | | |

The results of Example 4 show that treatment of guinea pigs with the viscous, low-pH composition of the present disclosure does not reduce the viral load in the nares to below limit of detection over 7 days (day −1, day 0, day 1, day 2, day 3, day 4, day 5 (the last day for which viral load matters in a transmission event for the transmission experiments), with inoculation by virus on day 0).

The results of the examples show that the viscous, low-pH composition reduces transmission of influenza virus between guinea pigs without reducing the level of virus in the nares. Without being bound by theory, it is believed that this surprising result may indicate that influenza virions may be able to replicate in the nares but not escape the film-former coated nasal mucosa, which can be disrupted during nasal washes to give viable virus that can form a plaque on MDCK cells.

1. A method of preventing transmission of a virus from a mammalian subject, the method comprising,
applying to a surface of a nasal cavity of the mammalian subject an aqueous composition comprising:
a surfactant; and
a hydroxycarboxylic acid;
wherein the aqueous composition is at least substantially free of an antimicrobial agent; and
wherein the aqueous composition has a viscosity of between 2,000 and 100,000 cps.

2. The method of claim 1, wherein the surfactant comprises a nonionic surfactant having an HLB value of at least 9.

3. The method of claim 1, wherein the surfactant comprises an anionic or amphoteric surfactant selected from the group consisting of sulfonates, sulfates, phosphates, phosphonates, and ammonium sulfonate amphoterics.

4. The method of claim 1, wherein the surfactant comprises an amine oxide.

5. The method of claim 1, wherein the surfactant is present in the aqueous composition in an amount of between 0.1 and 10 wt. %, based on the total weight of the aqueous composition.

6. The method of claim 1, wherein the hydroxycarboxylic is selected from the group consisting of lactic acid, malic acid, or citric acid.

7. The method of claim 1, wherein hydroxycarboxylic acids are present in the aqueous composition in an amount of between 1 and 20 wt. %, based on a total weight of the aqueous composition.

8. The method of claim 1, wherein the aqueous composition further comprises a cellulose-based thickening agent.

9. The method of claim 8, wherein the cellulose-based thickening agent is present in an amount of between 0.5 and 10 wt. %, based on a total weight of the aqueous composition.

10. The method of claim 1, wherein the aqueous composition has a pH of between 2.5 and 5.

11. The method of claim 1, wherein the aqueous composition has a pH of between 3 and 3.5.

12. The method of claim 1, wherein the aqueous composition, when subjected to the Time-Kill test, exhibits a log reduction of microbes no greater than 3 logs.

13. The method of claim 1, wherein the step of applying to a surface of the nasal cavity comprises inserting a swab having the aqueous composition borne on a surface thereof into the nasal cavity or spraying the aqueous composition into the nasal cavity.

14. A method of preventing transmission of a virus, the method comprising:
applying, to a mucosal tissue of a first mammalian subject and a second mammalian subject, an effective amount of an aqueous composition, wherein the first mammalian subject is substantially colonized by the virus and the second mammalian subject is not substantially colonized by the virus; and
allowing the first mammalian subject to interact with the second mammalian subject,
wherein the mucosal tissue is located in at least one of a nasopharynx, a nasal cavity, or an anterior nares, and
wherein the aqueous composition comprises:
a surfactant; and
a hydroxycarboxylic acid,
wherein the aqueous composition is substantially free of an antimicrobial agent; and wherein the aqueous composition has a viscosity of between 2,000 and 100,000 cps.

15. The method of claim 14, wherein applying the effective amount of the composition comprises applying the composition at least 24 hours before allowing the first and second mammalian subjects to interact.
